(19) United States
(12) Patent Application Publication
(10) Pub. No.: US 2025/0380731 A1
(43) Pub. Date: Dec. 18, 2025

(54) ENZYMATIC PROCESS FOR THE PRODUCTION OF A COMPOSITION RICH IN MALTOTETRAOSE

(71) Applicant: ROQUETTE FRERES, LESTREM (FR)

(72) Inventors: Pierre LANOS, LA BASSEE (FR); Jean Christophe CAILLIAU, SANTES (FR)

(21) Appl. No.: 18/867,650

(22) PCT Filed: May 25, 2023
(86) PCT No.: PCT/EP2023/025245

§ 371 Date: Sep. 4, 2025

(30) Foreign Application Priority Data
May 25, 2022 (FR) FR2205033

(51) Int. Cl. A23L 29/30; A23K 10/14; A23K 20/163; A23L 27/30; C12N 9/24; C12N 9/26

(52) U.S. Cl. CPC A23L 29/35; A23K 10/14; A23K 20/163; A23L 27/35; C12N 9/2402; C12N 9/2414; C12Y 302/01001; C12Y 302/0106

(57) ABSTRACT

The invention relates to an enzymatic process for preparing a composition rich in maltotetraose.

### TECHNICAL FIELD

The present invention relates to an enzymatic process for the production of a composition rich in maltotetraose.

The invention further relates to a composition rich in maltotetraose.

The present invention further relates to the use of a composition rich in maltotetraose for the preparation of foodstuffs for human or animal consumption.

### PRIOR ART

Maltotetraose is an oligosaccharide consisting of 4 glucose units linked linearly by alpha 1-4 glycosidic bonds.

Compositions rich in maltotetraose, or maltotetraose syrups, can be used in the manufacture of foodstuffs for human or animal consumption. Maltotetraose syrups offer many advantages over sucrose or glucose syrups. This is because they are less sweet than sucrose syrups, while preserving the taste of the food. In addition, they are less susceptible to Maillard degradation and have a higher viscosity.

Maltotetraose syrup is generally made from starches, such as corn starch, by enzymatic reactions. The starch is first hydrolyzed by an alpha-amylase to produce maltodextrins.

Maltodextrin compositions are mixtures of different sugars derived from starch hydrolysis and having different degrees of polymerization. This degree of polymerization or “DP” is reflected experimentally by the “dextrose equivalent”, or D.E., dextrose being D-glucose, the result of total starch hydrolysis. The higher the D.E., the more thorough the hydrolysis, and therefore the higher the proportion of simple (short-chain) sugars in the maltodextrin. A D.E. of zero would represent the starch itself, while a D.E. of 100 would represent pure dextrose, that is, a totally transformed starch.

The D.E. limit for maltodextrin is 20. Beyond that, the legal name of the resulting product is “glucose syrup”.

DE and DP are therefore inversely correlated.

The maltodextrins are then subjected to further enzymatic reactions to produce composition rich in maltotetraoses, that is, rich in DP4 oligosaccharides. Certain enzymes, such as maltotetrahydrolase from Pseudomonas saccharophila (“PS-4”) are capable of preferentially hydrolyzing maltodextrins to DP4.

However, conventional production of composition rich in maltotetraoses does not allow us to obtain compositions with maltotetraose proportions greater than or equal to 60%.

For example, patent U.S. Pat. No. 3,654,082 (CPC Internationale Inc) describes a process for producing maltotetraose syrup using the enzymatic activity of an amylase derived from Pseudomonas stutzeri

A maltotetrahydrolase enzyme is marketed by Genencor/DuPont under the name “OPTIMALT® 4G”.

In addition, international application WO2010/118269A2 (Danisco/DuPont) describes variants of the Pseudomonas saccharophila PS-4 enzyme with increased exo-alpha-amylase activity compared to the native enzyme and reduced endo-alpha-amylase activity compared to the native enzyme.

In application WO2010/118269A2, the syrups obtained in the examples have a maximum DP4 content of 47%.

WO2010132157 describes the use of the PS-4 enzyme with a pullulanase enzyme to form syrups comprising between 40 and 60% maltotetraose by weight.

In addition, Tereos has marketed a syrup called MYLOSE® 351, which is presented as a DP4-rich glucose syrup. However, this product comprises between 50 and 60% maltotetraose by weight. It also contains significant quantities of DP3 (between 6 and 12%)

It is therefore desirable to obtain compositions rich in maltotetraose and low in mono-, di- and tri-saccharides

### DETAILED DESCRIPTION OF THE INVENTION

The applicant company then found that it was possible to produce a composition rich in maltotetraose from an amylopectin-rich liquefied starch by enzymatic reaction. The company has thus developed a process that uses a particular enzyme, in combination with a tangential filtration step with a cut-off at 1 kDa, carried out simultaneously with the enzymatic reaction.

In a first aspect, the present invention relates to a process for preparing a composition comprising at least 60% maltotetraose by weight, said process comprising bringing a substrate into contact with a glucan 1,4-alpha-maltotetrahydrolysase enzyme and tangential filtration with a cut-off at 1 kDa carried out simultaneously with the enzymatic reaction.

According to a preferred embodiment of the invention, the process is a process for preparing a composition comprising at least 60%, preferably at least 65%, even more preferably at least 70%, at least 75%, at least 78%, at least 80% maltotetraose by weight, relative to the total dry weight of the composition.

The percentage of maltotetraose (also known as “DP4”) can be determined by any suitable method known to the person skilled in the art. For example, this percentage can be determined by HPLC chromatography, as shown in the examples below.

The substrate according to the process of the invention can be any product resulting from the partial hydrolysis of starch.

Preferably, the starting starch is an amylopectin-rich starch, such as waxy corn starch.

According to one embodiment of the invention, the substrate is selected from an amylopectin-rich liquefied starch and a low-DE maltodextrin.

Typically, the substrate has a DE between 2 and 15, preferably between 2 and 12.

Typically, the substrate comprises at least 90%, preferably at least 92%, at least 95%, even more preferably at least 95% by weight of amylopectin chains relative to the weight of dry matter of said substrate.

The process uses an enzyme of the glucan 1,4-alpha-maltotetrahydrolysase type (EC 3.2.1.60). This enzyme hydrolyzes alpha 1-4 glucosidic bonds every four residues, from the non-reducing ends of polysaccharides.

Preferably, it is a PS-4 variant described in patent application WO2010/118269.

Preferably, it is the enzyme marketed by Genencor/DuPont under the name “OPTIMALT® 4G”.

In one embodiment, the method according to the invention does not comprise the use of pullulanase.

In the process according to the invention, a tangential filtration step with a 1 kDa cut-off is carried out simultaneously with the enzymatic reaction. Low-molecular-weight sugars, particularly DP4s, are filtered out and collected as the enzymatic reaction progresses. The 1 kDa cut-off allows sugars of lower molecular weight (DP1, DP2, DP3, D4, DP5 and DP6) to pass into the filtrate, while retaining sugars of higher molecular weight (retentate).

Without wishing to be bound by any theory, the inventors have proposed that this simultaneous filtration shifts the reaction equilibrium in favor of DP4 production by removing this reaction product as it is produced.

In this way, the enzymatic reaction can continue until the residual substrate is a limiting dextrin, that is, essentially made up of alpha 1-6 glucosidic branched bonds.

In a preferred embodiment, filtration is started before the enzyme is added and a first volume of filtrate is discarded. This cleans the enzymatic reaction of any low DP sugars (mono-, di- and tri-saccharides) that may be present in the substrate.

Subsequently, once the enzyme has been added, the filtrate is collected. This filtrate contains mainly maltotetraose (or DP4), since the enzyme does not generate DP1, DP2 or DP3 sugars.

Thus, according to a particular embodiment, the invention relates to a process comprising the following steps:
supplying a substrate solution with a pH between 5 and 5.5
starting the filtration, keeping the retentate volume constant by adding demineralized water,
adding the enzyme
discarding the 1st filtrate fractions rich in monosaccharides, disaccharides and trisaccharides
collecting the following filtrate fractions enriched with maltotetraose,
if necessary, mixing and concentrating the maltotetraose-rich fractions,
if necessary, demineralizing the composition obtained
if necessary, freeze-drying the composition.

In a second aspect, the present invention relates to a composition obtainable by the process described above.

The present invention also relates to a composition comprising at least 60% maltotetraose by weight and less than 12% DP1, DP2 or DP3 oligosaccharides relative to the total weight of dry matter.

Thus, the cumulative weight of DP1, DP2 and DP3 in the composition according to the invention does not exceed 12%, preferably 11%, preferably 10%, preferably 9%, preferably 8%, preferably 7%.

According to a particular embodiment, the weight of DP1 in the composition according to the invention does not exceed 2%, preferably 1.8%, preferably 1.6%, preferably 1.4%, preferably 1.2%, preferably 1.0%.

According to a particular embodiment, the weight of DP2 in the composition according to the invention does not exceed 5%, preferably 4%, preferably 3%, preferably 2.9%, preferably 2.8%, preferably 2.7%, preferably 2.6%, preferably 2.5%.

According to a particular embodiment, the weight of DP3 in the composition according to the invention does not exceed 6%, preferably 5%, preferably 4%, preferably 3.8%, preferably 3.7%, preferably 3.6%, preferably 3.5%.

Finally, the present invention relates to the use of a composition according to the invention in the preparation of foodstuffs for human or animal consumption.

The invention will be better understood with the aid of the following examples, which are intended to be illustrative and non-limiting.

### Example 1: Protocol for Preparing a Maltotetraose-Rich Syrup

### 1. Reagents Used

1.1. Maltodextrin with a DE (dextrose equivalent) of 2 to 6 produced from Waxy corn starch (GLUCIDEX® 2 marketed by the applicant company).
1.2. Maltodextrin with a DE of 12 produced from standard corn starch (GLUCIDEX® 12 marketed by the applicant company).
1.3. Glucan 1,4-alpha-mltotetraohydrolylase enzyme: OPTIMALT®4G produced by Genencor (Dupont).
1.4. Heat-resistant Alpha amylase enzyme: LIQUOZYME® supra produced by Novozyme.
1.5. N200 ® waxy corn starch marketed by the applicant company.

Products 1.4 and 1.5 were only used in Example 4.

### 2. Equipment Used

The enzymatic reaction was carried out in a 2-liter beaker placed on a regulated heated magnetic stirrer.

Crossflow filtration was carried out simultaneously with the enzymatic reaction using a Centramate PALL system equipped with a 1 KD REF OS001T12 0.1 m2 nanofiltration cassette.

### 3. Procedure

The following procedure was used:
Prepare a maltodextrin solution by diluting 100 g of product in 900 ml of demineralized water in a 2-liter beaker.
Check pH of solution (5 to 5.5) and adjust if necessary with NaOH or HCl 0.1 mol/liter.
Shake the solution at 50° C.
Start nano-filtration with the Centramate system, allowing the filtrate to drain into a beaker. Once the 300-ml filtrate volume has been reached, add 200 μL of OPTIMALT® 4G enzyme to the maltodextrin solution and recycle the filtrate into the retentate for 15 minutes.
Take a grab sample every 300 mL of filtrate. On each sample, take the refractometry reading and analyze the levels of glucose (DP1), maltose (DP2), maltotriose (DP3), maltotetraose (DP4) and the higher levels of DP4 by HPLC.
The decrease in retentate volume must be continuously compensated with demineralized water (constant volume dialysis mode).
The fractions were mixed and concentrated on a rotary evaporator to a refractometric reading of 30 BX.
The resulting syrup was decolorized using SA activated carbon, the usual method for purifying glucose syrups (contact time: 1 H at 70° C.).
The syrup was then demineralized with the anionic and cationic resins normally used for glucose syrup purification.

If required, the syrup can be concentrated to a refractometric reading of 65-70 BX, or freeze-dried to preserve the sample.

### 4. Physical/Chemical Analysis

The carbohydrate composition of the fractions was analyzed using a Waters E2695 HPLC system equipped with a RID 2414 detector. The column used is an AMINEX HPX 87N at a temperature of 85°. Mobile phase flow rate (water): 0.3 mL/min.

### Example 2: Preparation of a Maltotetraose-Rich Syrup from a Low-DE Maltodextrin Produced from Waxy Starch

According to the protocol described in Example 1, the test described below was carried out with a solution of GLUCIDEX® 2 (100 g QSP demineralized water 1000 g) as starting substrate.

The various fractions of 300 ml of filtrate were recovered and the composition analyzed by HPLC. Fraction F1 corresponds to the filtrate before enzyme addition.

Fractions F4 to F8 were mixed for concentration and purification.

The results are presented in Table 1.

TABLE 1

| | Carbohydrate composition expressed in %/dry matter | | | | |
|---|---|---|---|---|---|
| | | | | | Oligo- |
| | | | | | saccha- |
| | Glucose | Maltose | Maltotriose | Maltotetraose | rides > |
| Reference | DP1 | DP2 | *DP3 | DP4 | DP4 |
| F1 (before | 1 | 6.4 | 10.1 | 6.7 | 75.6 |
| enzyme) | | | | | |
| F2 | 0.8 | 5.0 | 6.5 | 57.8 | 29.9 |
| F3 | 0.8 | 4.3 | 5 | 71.9 | 18.0 |
| F4 | 0.6 | 2.8 | 3.8 | 79.6 | 13.1 |
| F5 | 0.8 | 2.9 | 3.3 | 82.4 | 10.5 |
| F6 | 0.7 | 2.9 | 3.9 | 84.5 | 9.1 |
| F7 | 0.7 | 2.2 | 2.5 | 86.5 | 8.0 |
| F8 | 0.8 | 2.1 | 24 | 87.0 | 7.8 |
| Final mix | 0.7 | 2.5 | 3.2 | 83.7 | 9.9 |
| F4 to F8 | | | | | |

### Example 3: Preparation of a Maltotetraose-Rich Syrup from a DE 12 Maltodextrin Produced from Waxy Starch

According to the protocol described in Example 1, the test described below was carried out with a solution of GLUCIDEX® 12 (100 g QSP demineralized water 1000 g) as starting substrate.

The various fractions of 300 ml of filtrate were recovered and the composition analyzed by HPLC. Fraction F0 corresponds to the filtrate before enzyme addition.

The results are presented in Table 2.

TABLE 2

| | Carbohydrate composition expressed in %/dry matter | | | | |
|---|---|---|---|---|---|
| | | | | | Oligo- |
| | | | | | saccha- |
| | Glucose | Maltose | Maltotriose | | rides > |
| Reference | DP1 | DP2 | *DP3 | MaltotetraoseDP4 | DP4 |
| F0 (before | 1.9 | 7.9 | 12.4 | 10.4 | 67.4 |
| enzyme) | | | | | |
| F1 | 1.9 | 7.7 | 11.3 | 29.9 | 19.3 |
| F2 | 1.9 | 6.9 | 10.1 | 45.7 | 35.4 |
| F3 | 1.9 | 6.6 | 9.5 | 51.5 | 30.5 |
| F4 | 1.9 | 5.7 | 8.2 | 61.5 | 22.9 |
| F6 | 1.7 | 4.0 | 5.9 | 71.5 | 16.9 |
| F7 | 1.5 | 3.0 | 5.0 | 65.5 | 25.0 |
| F8 | 1.7 | 3.1 | 4.9 | 57.7 | 32.6 |

The aim of the trial was to compare the richness of maltotetraose fractions as a function of raw material. The product was not purified.

### Example 4: Preparation of a Maltotetraose-Rich Syrup from Liquefied Waxy Corn Starch

In contrast to the two previous examples, the raw material for the trial was a waxy corn starch liquefied as follows:
Preparation of a Waxy corn starch suspension (100 g q.s. demineralized water 400 g)
Liquefaction with heat-resistant alpha-amylase (LIQUOZYME® as above) at a concentration of 0.8 g/kg dry matter.
Microwave (5 1-minute cycles, 1000 w) then hold at 95° C. for 15 min.
Inhibit with 1 N HCl up to pH=3.0
Filter on COFRAM filter ref. BECO KD3
Dilute the resulting solution to obtain 1 liter of 10% dry matter solution.

The composition of the resulting liquefied corn starch is shown in Table 3.

TABLE 3

| | Carbohydrate composition expressed in %/dry matter | | | | |
|---|---|---|---|---|---|
| | | | | | Oligo- |
| | | | | | saccha- |
| | Glucose | Maltose | Maltotriose | | rides > |
| Reference | DP1 | DP2 | *DP3 | MaltotetraoseDP4 | DP4 |
| Liquefied | 0.1 | 1.0 | 2.1 | 0.9 | 95.8 |
| corn | | | | | |
| starch | | | | | |

From the carbohydrate composition, we can estimate that the DE (dextrose equivalent) of the resulting solution is around 6.

This solution was then treated according to the conditions described in Example 1.

The following results were obtained:

TABLE 4

| | Carbohydrate composition expressed in %/dry matter | | | | |
|---|---|---|---|---|---|
| | | | | | Oligo- |
| | | | | | saccha- |
| | Glucose | Maltose | Maltotriose | | rides > |
| Reference | DP1 | DP2 | *DP3 | MaltotetraoseDP4 | DP4 |
| F1 (before | 1.1 | 7.3 | 15.3 | 8.8 | 66.7 |
| enzyme) | | | | | |
| F2 | 0.9 | 5.2 | 9.6 | 53.6 | 30.6 |
| F3 | 1.0 | 3.7 | 6.6 | 71.2 | 17.5 |
| F5 | 0.8 | 2.9 | 2.9 | 77.4 | 13.6 |
| F6 | 0.8 | 2.2 | 3.9 | 80.0 | 13.1 |
| F7 | 0.7 | 2.4 | 4.1 | 78.2 | 14.8 |

The aim of the trial was to compare the richness of maltotetraose fractions as a function of raw material. The product was not purified.

All the above examples show that the process according to the invention makes it possible to obtain compositions rich in maltotetraose and low in DP1, DP2 and DP3, from three different types of substrate (maltodextrin with DE2, maltodextrin with DE 12 and liquefied starch with DE about 6)

The invention claimed is:

1. A process for preparing a composition comprising at least 60% maltotetraose by weight, said process comprising bringing a substrate into contact with a glucan 1,4-alpha-maltotetrahydrolysase enzyme and tangential filtration with a cut-off at 1 kDa carried out simultaneously with the enzymatic reaction.

2. The process according to claim 1, wherein the substrate is selected from a amylopectin-rich liquefied starch or a low-DE maltodextrin.

3. The process according to claim 1, wherein said process does not include the use of a pullulanase.

4. The method according to claim 1, comprising the following steps:
supplying a substrate solution with a pH between 5 and 5.5
starting the filtration, keeping the retentate volume constant by adding demineralized water,
adding the enzyme
discarding the 1st filtrate fractions rich in monosaccharides, disaccharides and trisaccharides,
collecting the following filtrate fractions enriched with maltotetraose,
if necessary, mixing and concentrating the maltotetraose-rich fractions,
if necessary, demineralizing the composition obtained
if necessary, freeze-drying the composition.

5. A composition that can be obtained by the method according to claim 1.

6. A composition comprising at least 60% maltotetraose by weight and less than 12% DP1, DP2 or DP3 oligosaccharides.

7. The use of a composition according to claim 5 for preparing foodstuffs for human or animal consumption.
